(19) United States
(12) Patent Application Publication
(10) Pub. No.: US 2024/0094099 A1
(43) Pub. Date: Mar. 21, 2024

(54) Magnetic Particle Air Transfer

(71) Applicant: Abbott Diagnostics Scarborough, Inc., Scarborough, ME (US)

(72) Inventors: Austin M. Derfus, Solana Beach, CA (US); Karthikeyan Kumaravadivelu, San Diego, CA (US); Kai Yang, San Diego, CA (US); Adrian P. Crute, Escondido, CA (US); Darren S. Gray, San Diego, CA (US); Armando R. Tovar, San Diego, CA (US); Tomoko Kobayashi, Osaka (JP); Tomotaka Komori, Osaka (JP); Yoshiko Wada, Osaka (JP); Masato Orikasa, Osaka (JP); Toru Yoshimura, Osaka (JP)

(21) Appl. No.: 18/274,325

(22) PCT Filed: Jan. 28, 2022
(86) PCT No.: PCT/US2022/014422

§ 371 Date: Jul. 26, 2023

Related U.S. Application Data
(60) Provisional application No. 63/143,494, filed on Jan. 29, 2021.

(51) Int. Cl. G01N 1/34; C12N 15/10

(52) U.S. Cl. CPC G01N 1/34; C12N 15/1013

(57) ABSTRACT

The methods for sample preparation disclosed herein utilize an air phase to reduce aqueous phase associated with magnetic particles bound to nucleic acids to decrease carryover of one or more contaminants, such as, cell debris, chaotropic agents, non-specifically attached molecules, and the like. This air-transfer step is improved by using a combination of a first population of magnetic particles and a second population of magnetic particles, where the first population of magnetic particles is capable of associating with nucleic acids, and the magnetic particles in the second population are at least two-times larger in size than the size of the magnetic particles in the first population. As discussed herein, the use of this second population of magnetic particles improves the transfer of the first population of magnetic particles by reducing loss of the first magnetic particles during the transfer. The sample preparation methods may be semi-automated or completely automated.

### CROSS-REFERENCE TO RELATED APPLICATIONS

This application claims the benefit of U.S. Provisional Patent Application No. 63/143,494, filed Jan. 29, 2021, which application is incorporated herein by reference in its entirety.

### INTRODUCTION

Nucleic acid isolation and purification is a set of molecular biology techniques used for the extraction of DNA and RNA for use in downstream applications. Nucleic acid isolation and purification approaches include column-based isolation and purification, reagent-based isolation and purification, magnetic bead-based isolation and purification, and other technologies. Reagents, kits and instruments that find use in isolating and purifying nucleic acids are available. Poor sample preparation can lead to suboptimal results in downstream applications, and it is for this reason that optimized versions of kits have emerged to address variation in sample source, be it blood, plant tissue, fungi, or bacteria.

A sample preparation process includes releasing a nucleic acid target from its native biological source (e.g., lysis of cells, such as patient cells or lysis of microorganisms, such as, virus, bacteria, fungi, etc.) using chaotropic nucleic acid extraction technology, binding of nucleic acids to a solid phase (e.g., paramagnetic particles) using silica or iron oxide nucleic acid chemistry, separation of the solid phase from the residual lysis solution using magnetic separation technology, washing to remove unwanted materials, and elution or separation of nucleic acid from the solid phase using fluid handling technology. At the completion of the sample preparation protocol, the sample is transferred to a PCR component of a device for nucleic acid detection.

### SUMMARY

Aspects of the present disclosure include sample preparation methods, sample preparation cartridges, and sample preparation systems.

The methods for sample preparation disclosed herein utilize a step of transferring magnetic particles bound to an analyte through an air phase. This transferring step is referred to as air-transfer. The air-transfer step reduces carryover of aqueous phase with magnetic particles. Reduction of carryover of aqueous phase with magnetic particles decreases contaminants, such as, cell debris, chaotropic agents, non-specifically attached molecules, and the like. This air-transfer step is improved by using a combination of a first population of magnetic particles and a second population of magnetic particles, where the first population of magnetic particles is capable of associating with a target analyte, and the magnetic particles in the second population are at least two-times larger in size than the size of the magnetic particles in the first population. As discussed herein, the use of this second population of magnetic particles improves the transfer of the first population of magnetic particles by reducing loss of the first magnetic particles during the air-transfer. The sample preparation methods may be semi-automated or completely automated.

Also provided herein are sample preparation cartridges that include a first chamber comprising the first population of magnetic particles and the second population of magnetic particles and a second chamber configurable as an air chamber and adjacent the first chamber, where the magnetic particles may optionally be present in a pellet form and may optionally be in a lyophilized form.

The systems for sample preparation provided herein include the disclosed sample preparation cartridges and a magnet operably placed in association with the sample preparation cartridge such that the magnet can exert a magnetic force on the magnetic particles present in the sample preparation cartridge. The system may optionally include an instrument that comprises a processor comprising instructions for performing one or more steps of the disclosed methods.

### BRIEF DESCRIPTION OF THE FIGURES

FIGS. 1A-1B shows schematics of sample preparation cartridges and reagents for performing the disclosed methods.

FIGS. 1C-1D shows sample preparation cartridges with three connected chambers. An air chamber is positioned between two chambers comprising aqueous phases. A magnet operably positioned adjacent the first chamber is shown in FIG. 1D.

FIG. 2 shows schematics for a sample preparation cartridge and method steps for sample preparation according to one embodiment of the present disclosure.

FIG. 3 shows schematics for a sample preparation cartridge and method steps for sample preparation according to another embodiment of the present disclosure.

FIG. 4A shows a cylindrical sample preparation cartridge according to one embodiment of the present disclosure.

FIG. 4B shows a zoomed-in image of lower region of a cylindrical sample preparation cartridge according to one embodiment of the present disclosure.

FIG. 4C shows a further zoomed-in image of the lower region of the cylindrical sample preparation cartridge according to one embodiment of the present disclosure.

FIG. 4D shows a cylindrical sample preparation cartridge according to one embodiment of the present disclosure.

FIGS. 4E-4F shows a sample preparation cartridge with the film forming a side wall of the chambers removed to show the chambers and channels positioned on the annular wall.

FIG. 4G shows chamber 103 with a shelf-baffle 108 extending transversely through the chamber.

FIG. 4H shows a chamber 103 with a bottom wall raised at one end.

FIG. 5 shows a sample preparation system that includes a sample preparation cartridge 100 and a cylinder housing 130, according to one embodiment of the present disclosure.

FIG. 6 shows an illustration of interfacial boundary between the air phase in the middle chamber and the aqueous phases in two chambers adjacent the middle chamber.

FIG. 7 shows the results obtained from using air or an oil as the immiscible phase.

### DETAILED DESCRIPTION

Aspects of the present disclosure include sample preparation methods, sample preparation cartridges, and sample preparation systems.

The methods for sample preparation disclosed herein utilize an air phase to reduce aqueous phase associated with magnetic particles bound to nucleic acids to decrease carryover of one or more contaminants, such as, cell debris, chaotropic agents, non-specifically attached molecules, and the like. This air-transfer step is improved by using a combination of a first population of magnetic particles and a second population of magnetic particles, where the first population of magnetic particles is capable of associating with nucleic acids, and the magnetic particles in the second population are at least two-times larger in size than the size of the magnetic particles in the first population. As discussed herein, the use of this second population of magnetic particles improves the transfer of the first population of magnetic particles by reducing loss of the first magnetic particles during the transfer. The sample preparation methods may be semi-automated or completely automated.

Before the present sample preparation cartridge and methods are described in greater detail, it is to be understood that the present disclosure is not limited to particular embodiments described, as such may, of course, vary. It is also to be understood that the terminology used herein is for the purpose of describing particular embodiments only, and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the present sample preparation cartridges, methods, and sample preparation units. The upper and lower limits of these smaller ranges may independently be included in the smaller ranges and are also encompassed within the sample preparation cartridges, methods, and sample preparation units, subject to any specifically excluded limit in the stated range. Where the stated range includes one or both of the limits, ranges excluding either or both of those included limits are also included in the sample preparation cartridges, methods, and sample preparation units.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating un-recited number may be a number which, in the context in which it is presented, provides the substantial equivalent of the specifically recited number.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present sample preparation cartridges, methods, and sample preparation units, representative illustrative sample preparation cartridges, methods, and sample preparation units are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present sample preparation cartridges, methods, and sample preparation units. Any recited method can be carried out in the order of events recited or in any other order which is logically possible.

### Method for Sample Preparation

As summarized above, aspects of the present disclosure include method for sample preparation. The phrases “sample preparation” and “sample processing” are used herein interchangeably and refer to the process for isolating a target analyte, e.g., a cell, virus, protein, or nucleic acids present in a sample. The process involves binding of the target analyte present in an aqueous phase to magnetic particles capable of binding to the analyte; an air-transfer step that reduces aqueous phase associated with the magnetic particles; and an elution step that releases the target analyte bound to the magnetic particles. As discussed in detail elsewhere, the sample may be preprocessed prior to binding to the magnetic particles. When the sample does not include free target analyte, e.g., protein or nucleic acids, i.e., where the protein or nucleic acids are present in a microorganism or a cell, the sample preparation methods disclosed herein may involve sample lysis to release the protein or nucleic acids into an aqueous solution.

According to certain embodiments, the method for processing the sample may include contacting a sample with a first population of magnetic particles and a second population of magnetic particles in an aqueous phase in a first region of a sample preparation cartridge. The first population of magnetic particles is capable of associating with the target analyte, and the magnetic particles in the second population are at least two-times larger in size than the size of the magnetic particles in first population. The method further comprises transporting the first and second populations of magnetic particles from the aqueous phase in the first region to an air phase in a second region of the cartridge by applying a magnetic force to the magnetic particles. The term “capable of associating” as used herein in the context of magnetic particles and an analyte means that the magnetic particles can bind to the analyte. The magnetic particles can be functionalized using standard methods such that the magnetic particles can bind to a target analyte. The functionalization is sufficiently specific under conditions, such as, presence of a lysis buffer. For example, the magnetic particles may be functionalized to bind to nucleic acids or to proteins. In certain examples, nucleic acids may be attached to surface of PMPs by silica or iron oxide nucleic acid chemistry.

The magnetic particles in the first population may have a diameter of 500 nm-10 um. For example, the average diameter of the particles in the first population may be about 500 nm-3 um, 2 um-6 um, 4-7 um, or 8-10 um. The magnetic particles in the second population may have a diameter that is 2×-20× the diameter of the magnetic particles in the first population, such as, 2×-10×, 3×-10×, or 3×-5× the diameter of the magnetic particles in the first population. For example, the average diameter of the particles in the first population may be 1 um-3 um and the average diameter of the particles in the second population may be 6-60 um, e.g., 9 um-50 um, 10 um-30 um, or 10 um-20 um. In other examples, the average diameter of the particles in the first population may be 2 um-6 um and the average diameter of the particles in the second population may be 10-60 um, e.g., 10 um-50 um, 10 um-30 um, or 10 um-20 um. In other examples, the average diameter of the particles in the first population may be 3 um-7 um and the average diameter of the particles in the second population may be 10 140 um, e.g., 10 um-120 um, 20 um-120 um, or 50 um-120 um. In yet another example, the average diameter of the particles in the first population may be 8 um-10 um and the average diameter of the particles in the second population may be 20-60 um, e.g., 20 um-50 um, or 20 um-30 um.

The ratio of the amount of the first and second magnetic particles may be a ratio of 1:1, 2:1, 1:2, 3:1, 1:3, 1:10, 1:30, 1:100, and so on. In certain cases, the ratio of the amount of the first and second magnetic particles may be 1:1. As used herein, amount refers to mass of the magnetic particles. The amount of the magnetic beads in the second population can vary. It can be as low as 1% or 99% of the total bead mass per reaction.

As used herein, magnetic particles refer to particles that are magnetically responsive. Magnetically responsive particles include or are composed of magnetically responsive materials. Examples of magnetically responsive materials include paramagnetic materials, ferromagnetic materials, ferrimagnetic materials, and metamagnetic materials. Examples of suitable paramagnetic materials include iron, nickel, and cobalt, as well as metal oxides, such as Fe₃O₄, BaFe₁₂O₁₉, CoO, NiO, Mn₂O₃, Cr₂O₃, and CoMnP. The first and second magnetic particles may each contain the same magnetically responsive material. For example, both types of particles may be made of the same paramagnetic material, such as, iron oxides, e.g., ferromagnetic and/or ferrimagnetic material, cobalt oxides, nickel oxides, and mixtures thereof. In certain aspects, the first and second magnetic particles do not include substantial amounts of non-magnetic elements, i.e., elements that do not become polarized in a magnetic field and are thus not attracted to the magnets. Such non-magnetic elements include second silver, gold, copper, and the like. In certain embodiments, the magnetic particles of the first and second populations of magnetic particles comprise a paramagnetic material enclosed in a non-magnetic polymer, such as, magnetic materials covered with a polymeric material or magnetic material embedded in a polymer matrix. Such particles may be referred to as magnetic or paramagnetic beads. The magnetic particles in the second population of magnetic particles (also referred to as “second magnetic particles”) may include more of the paramagnetic material resulting in a larger diameter as compared to the magnetic particles in the first population of magnetic particles (also referred to as “first magnetic particles”). As a result of the size difference, the second magnetic particles experience a stronger pull from the magnetic field as compared to the first magnetic particles. However, due to their closed proximity to the first magnetic particles, the second magnetic particles are able to pull along the first magnet particles, thus increasing the overall magnetic pull on the first magnetic particles.

In certain aspects, the second magnetic particles may be paramagnetic while the first magnetic particles may be superparamagnetic. In other aspects, the second magnetic particles may be superparamagnetic while the first magnetic particles may be paramagnetic. In some examples, the first population of magnetic particles may include magnetic particles covered with silica. In some examples, the second population of magnetic particles may include magnetic particles covered with agarose or sepharose or polystyrene. The magnetic particles may be magnetic microparticles and nanoparticles and/or superparamagnetic microparticles and nanoparticles.

While the size of the first and second magnetic particles are described in terms of diameter, the particles may not necessarily be spherical in size and can be amorphous in shape. For amorphous particles, the diameter is the largest distance from one side to the diametrically opposite side. In certain embodiments, the magnetic particles may be substantially spherical. Exemplary magnetic particles include those available from commercial sources, such as, Dynabeads® Magnetic beads provided by Invitrogen, Estapor® SuperParamagnetic Microspheres and PureProteome™ Magnetic Beads by Merck Millipore, BcMag™ by Bioclone Inc., ProMag™ and BioMag® from Bangslabs, SupraMag™ by Polymicrospheres Inc., TurboBeads® by Turbobeads Llc., and SPHERO™ Polystyrene Magnetic Particles by Spherotech and the like. Superparamagnetic beads are available from Sigma-Aldrich and Thermo Scientific.

The first and second magnetic particles may be functionalized to bind to a target analyte, e.g., nucleic acids, such as, DNA and RNA. In certain embodiments, only the first magnetic particles are functionalized to bind to the target analyte while the second magnetic particles do not appreciably bind to the target analyte. In some aspects, using the smaller diameter magnetic particles functionalized to bind to the target analyte reduces non-specific binding by other molecules present in the sample.

According to certain embodiments, the method may further involve transporting the first and second populations of magnetic particles from the air phase in the second region to an aqueous phase in a third region of the cartridge by applying a magnetic force to the magnetic particles.

In certain examples, applying the magnetic force causes the magnetic particles to aggregate in an area in the first region, which area is adjacent to the source of the magnetic force and the transporting involves maintaining the magnetic force on the aggregated magnetic particles, moving the aggregated magnetic particles to the air phase in the second region of the cartridge, and moving the aggregated magnetic particles to the aqueous phase in the third region of the cartridge.

In some cases, transporting the first and second populations of magnetic particles comprises moving a magnet generating the magnetic force relative to the different regions of the cartridge. In other cases, transporting the first and second populations of magnetic particles comprises moving the cartridge or a portion thereof relative to a magnet generating the magnetic force.

As described in more detail in the next section, the disclosed methods are not dependent on a particular configuration of the sample preparation cartridge. While exemplary configurations for the sample preparation cartridges are described, other configurations may also be used.

In certain example, the sample preparation cartridge may be a planar sample preparation cartridge. For example, the cartridge may be rectangular or circular but have a low profile such that it is substantially flat. In another example, the cartridge may be substantially cylindrical. In yet another example, the cartridge may include a first region that is a chamber and a separable second region that is a plate, this cartridge being used in conjunction with another cartridge that includes a third region to which the magnetic particles are transferred via the plate.

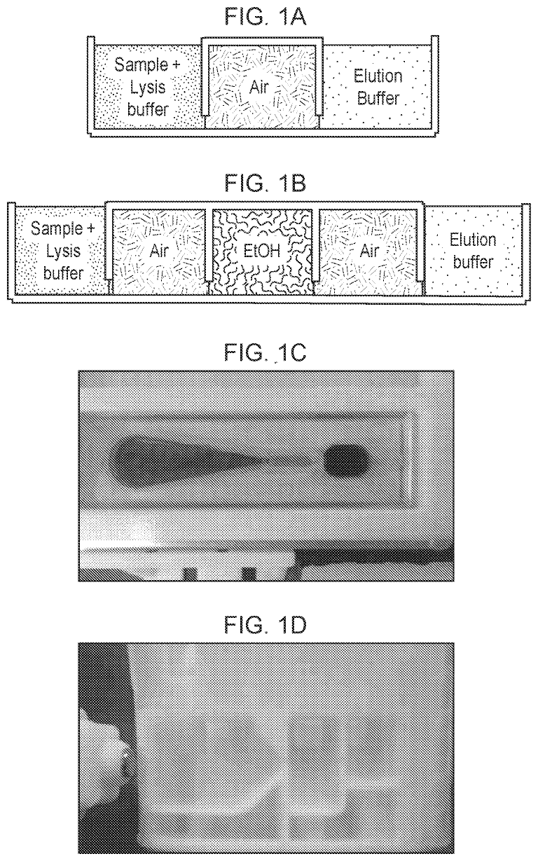

In one example, the cartridge is substantially planar and comprises a first plate placed in a spaced apart manner from a second plate, wherein the first and second plates are held in stationary position relative to each other. In certain embodiments, the cartridge is substantially planar and comprises a first plate placed in a spaced apart manner from a second plate, wherein the first and second plates are movable relative to each other such that the plates are held in a spaced apart and slidably movable configuration. The cartridge may be substantially planar and may not include separate chambers and the aqueous phase in the first region may be an aqueous droplet and the air phase in the second region is air present between the first and second plates, and if present, the aqueous phase in the third region is an aqueous droplet. Sample preparation devices having one or more of the features of such cartridges are described in U.S. Pat. No. 9,766,166, which is incorporated herein by reference. See, for example, FIGS. 1A-1G of U.S. Pat. No. 9,766,166.

According to certain embodiments, the first region is a first chamber comprises the aqueous phase and the second region is a second chamber comprises the air phase. The first and second chambers are connected via a first channel, wherein a difference in pressure between the first and second chambers establishes a liquid-air interface in the first channel. In certain example, the cartridge comprises a third region, where the third region is a third chamber comprising the aqueous phase.

In yet another embodiment, the disclosed method comprises transporting the first and second populations of magnetic particles from the air phase in the second region to an aqueous phase in a separate sample preparation cartridge, wherein the transporting comprises maintaining a magnetic force on the magnetic particles till the second region is in association with the aqueous phase in the separate cartridge and removing the magnetic force, thereby allowing the magnetic particles to be released into the aqueous phase. The first and second regions of the cartridge may be removably associated. For example, the first region is a first chamber, the second region is a transfer plate and the third region is a third chamber. Sample preparation devices having one or more of the features of such cartridges are described in U.S. Pat. No. 10,040,062, which is incorporated herein by reference. See, for example, FIGS. 1-5 of U.S. Pat. No. 10,040,062.

According to certain embodiments, the method for sample processing may include contacting a sample with a first population of magnetic particles and a second population of magnetic particles in a first chamber of a sample processing cartridge, where the first population of magnetic particles is capable of associating with the target analyte, e.g., nucleic acids, and the magnetic particles in the second population are at least two-times larger in diameter than the diameter of the first population of magnetic particles; transporting the first and second populations of magnetic particles from the first chamber to a second chamber of the cartridge by applying a magnetic force to the particles, where the second chamber comprises air and the first chamber comprises an aqueous solution, the first and second chambers are connected via a first channel, a difference in pressure between the first and second chambers establishes a liquid-air interface in the first channel; and transporting the first and second populations of magnetic particles from the second chamber to a third chamber comprising an aqueous solution by applying a magnetic force to the particles, where the second and third chambers are connected via a second channel, and a difference in pressure between the second and third chambers establishes an air-liquid interface in the second channel. Exemplary cartridge that may be used in such a method are described further in the next section and are depicted schematically in FIGS. 1A-1B and shown in FIGS. 1C-1D.

The contacting may be carried out under conditions sufficient for binding to the target analyte (e.g., nucleic acids) present in the sample to at least the first population of magnetic particles. In certain embodiments, the first chamber of the sample processing cartridge may include an aqueous phase in which the magnetic particles are present and can bind to the target analyte. In certain embodiments, the aqueous phase may be a lysis buffer. The lysis buffer may be a standard lysis buffer as is known in the art. For example, the lysis buffer may include chaotropic agents that cause lysis of microorganisms, such as bacteria, virus, etc. as well as cells, such as, mammalian cells. In certain examples, guanidine hydrochloride may be used as a chaotropic agent.

The step of contacting may be optionally involve agitating the mixture of the sample and an aqueous phase comprising the first and second populations of magnetic particles, where, optionally, the aqueous phase may comprise a lysis buffer. The contacting may be carried out for a period of time sufficient for the nucleic acids to bind to at least the first population of magnetic particles.

In some embodiments, the contacting the sample with the first and second populations of magnetic particles comprises contacting an aqueous solution comprising the first and second populations of magnetic particles with the sample. In some embodiments, the contacting comprises placing the sample in the first chamber followed by introducing into the first chamber the first and second populations of magnetic particles and aqueous solution. In some embodiments, the first chamber comprises the first and second populations of magnetic particles and introducing the aqueous solution wets the magnetic particles and causes dispersion of the magnetic particles. In some embodiments, the first chamber comprises a compartment comprising the first and second populations of magnetic particles and the introducing the aqueous solution wets the magnetic particles and causes the magnetic particles to flow from the compartment into the first chamber. The aqueous solution may be a lysis buffer. A lysis buffer may be used for processing samples that do not include free nucleic acid and includes nucleic acid that is present inside a cell or virus.

Many types of sample can be processed using the methods, cartridge and systems of the present disclosure. The sample includes or is suspected of including a material of interest comprising a cell, a virus, a protein, or a nucleic acid. In certain examples, the material of interest may be a nucleic acid present in a cell or a virus. In certain embodiments, the contacting results in disruption of a cell or virus present in the sample to release the nucleic acid present in the cell or virus, respectively.

In certain embodiments, the contacting comprises agitating a mixture comprising the sample and the first and second populations of magnetic particles. The agitating may be used to facilitate cell/virus lysis and/or ensure uniform dispersion of the magnetic particles. In certain embodiments, agitating may include shaking the cartridge. Shaking may be accomplished using a rotary shaker or a vortexer. In certain aspects, the sample preparation cartridge may be cylindrical in shape and may be rotated by back and forth motion about a central axis extending between the top and bottom ends of the cylinder.

In certain embodiments, the method comprises transporting the first and second populations of magnetic particles from the first chamber to a second chamber of the cartridge by applying a magnetic force to the particles. The second chamber of the sample processing cartridge may be filled with air, e.g., compressed air. The compressed air may be generated by filling of the first and third chambers with aqueous solution at atmospheric pressure. For example, at the start of the method, all three chambers may be empty and thus only have air. During sample processing, the first and third chambers are filled with an aqueous phase. The aqueous phase forces the air present in the first and third chambers into the second (middle) chamber, resulting in compression of the air present in the second chamber. The aqueous solution also flows into and partially fills the first channel extending between the first and second chambers and the second channel extending between the second and third chambers. The interface formed in the first and second channels between the aqueous and gas phases serves as a barrier that prevents aqueous matter from leaving the aqueous phase and entering the gas phase. Thus, the interface reduces the carryover of aqueous solution captured on or in between magnetic beads from the first chamber to the second chamber.

The method further comprises transporting the first and second populations of magnetic particles from the second chamber to a third chamber of the cartridge by applying a magnetic force to the particles. In certain embodiments, the third chamber comprises an aqueous phase which may be a wash buffer or an elution buffer. In certain embodiments, the third chamber comprises an elution buffer. In certain embodiments, the third chamber comprises a wash solution and the cartridge comprises a fourth chamber comprising air or an immiscible substance and a fifth chamber comprising an elution buffer. The sample processing method further includes transporting the first and second populations of magnetic particles from the third chamber to the fifth chamber via the fourth chamber.

In some embodiments, the transporting the first and second populations of magnetic particles from one chamber to an adjacent chamber of the cartridge by applying a magnetic force to the particles comprises placing a magnet adjacent the chamber to cause formation of an aggregate comprising the magnetic particles, wherein the magnet is placed at a position such that the aggregate is spatially aligned with the entrance to the first and second channels.

Transporting the first and second populations of magnetic particles may include moving a magnetic field relative to the cartridge, while the cartridge remains stationary; moving the cartridge relative to a stationary magnetic field, and/or moving the devi cartridge ce and the magnetic field. In certain aspects, the method may involve aggregating the magnetic particles after the contacting step by exposing the magnetic particles to a magnet and using the magnet to transport the aggregated magnetic particles. In certain embodiments, the method involves applying magnetic force to the magnetic particles to forms an aggregate of the first and second populations of magnetic particles which aggregate is spatially aligned with an entrance to the first channel. In other words, the magnet is positioned relative to the sample preparation cartridge such that the magnetic particles move to an area in the cartridge that is adjacent the magnet, which area is spatially aligned with an entrance to the first channel. In general, the area where the magnetic particles aggregate is the interior surface of a wall of the sample preparation cartridge, e.g., the wall forming one of the sides of a first, second, third chambers and the first and second channels. In certain embodiments, the first chamber of the sample preparation cartridge has a side that decreases in size from the first chamber to the first channel and facilitates transport of the aggregated particles from the first chamber to the second chamber via the first channel. This tapered entrance to the first channel can facilitate transport of not only the magnetic particles that are compactly aggregated adjacent the magnet but also loosely aggregated magnetic particles that may lag when the magnetic force moves the compactly aggregated magnetic particles.

As previously noted, the placement of the magnet may be such that the aggregate is spatially aligned with an entrance to the second channel, so no further movement of the aggregated magnetic particles is needed to move them to the entrance. In other cases, the aggregated magnetic particles may be moved by the magnetic force to line-up the aggregate to the entrance of the first channel.

In certain embodiments, the entrance to the second channel comprises a tapered region that decreases in size from the second chamber to the second channel and facilitates transport of the aggregate from the second chamber to the third chamber via the second channel.

In some embodiments, the transporting the first and second populations of magnetic particles from the first chamber to a second chamber of the cartridge by applying a magnetic force to the particles comprises placing a magnet adjacent the first chamber to cause formation of an aggregate comprising the magnetic particles, wherein the magnet is placed at a position such that the aggregate is spatially aligned with the entrance to the first and second channels.

According to certain embodiments, an external magnet may be placed at a distance of no more than 1 cm, no more than 9 mm, no more than 8 mm, no more than 7 mm, no more than 6 mm, no more than 5 mm, no more than 4 mm, no more than 3 mm, no more than 2 mm, no more than 1.5 mm, or no more than 1 mm, or no more than 0.5 mm, or no more than 0.2 mm, or no more than 0.1 mm from an outer surface of the cartridge which outer surface forms a wall of the chamber/region in which the magnetic particles are present. In certain examples, the external magnet may contact the outer surface of the cartridge which outer surface forms a wall of the chamber/region in which the magnetic particles are present. In certain example, the wall of the chamber/region in which the magnetic particles are present may have a thickness of less than 2 mm, less than 1 mm, less than 0.5 mm, e.g., a thickness of 0.1 mm-5 mm or 0.2 mm-4 mm, or 0.5 mm-2 mm.

In some embodiments, the method is semi-automatic. For example, at least one step of the method may be carried out by an instrument, instead of the user. In certain embodiments, the step of contacting a sample with a first population of magnetic particles and a second population of magnetic particles in a first chamber of a sample processing cartridge comprises loading of the sample into the first chamber of the sample processing cartridge by a user and wherein one or more of the remainder of the steps are carried out automatically. In some cases, the steps of agitating the mixture of sample and first and second populations of magnetic particles may be carried out automatically. In some cases, the steps transporting the magnetic particles may be automated. Automation may be achieved by, e.g., a computer that controls movement of the sample preparation cartridge and/or the magnet.

In certain embodiments, the method may further include, aggregating the magnetic particles in the third chamber (or fifth chamber) which is filled with an aqueous solution, such as, an elution buffer. This chamber may also be referred to as an elution chamber. Once the magnetic particles are aggregated and prevented from moving, the aqueous solution (e.g., elution buffer—such as a PCR amplification buffer) containing the nucleic acid isolated from the sample may be removed. The aqueous solution may be removed by pipetting it out manually or automatically. The aqueous solution may be removed by draining it from the third (or fifth chamber) by e.g., forcing it through a hole. The elution chamber may be connected to a collection container into which the aqueous solution can be drained into.

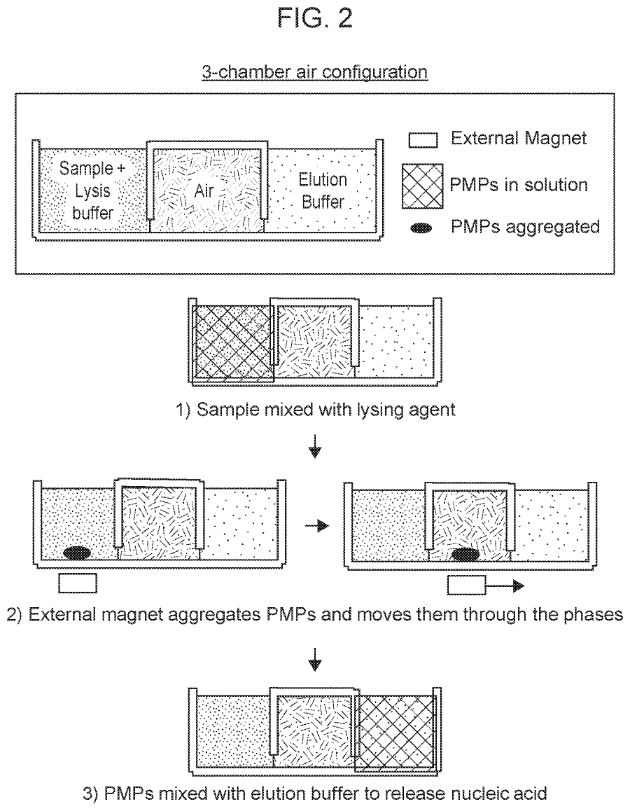

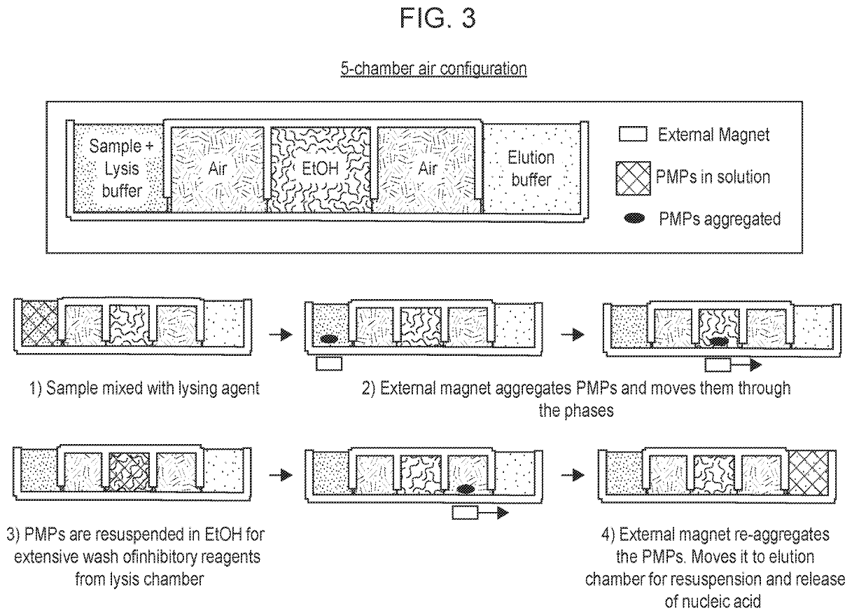

Exemplary methods are depicted schematically in FIGS. 2 and 3. FIG. 2 illustrates a sample preparation method that uses a sample processing cartridge comprising three chambers. The method includes mixing a sample with lysis buffer or lysing agent and paramagnetic particles (PMPs) such that the PMPs are in solution (Step 1). The PMPs are aggregated by bringing an external magnet in proximity to the cartridge and the aggregated PMPs are transported from the aqueous phase comprising the lysis buffer to air phase (Step 2). In step 3, the aggregated PMPs are transported from the air phase to the chamber comprising elution buffer.

FIG. 3 illustrates a sample preparation method that uses a sample processing cartridge comprising five chambers. The method includes mixing a sample with lysis buffer and paramagnetic particles (PMPs) such that the PMPs are in solution (Step 1). The PMPs are aggregated by bringing an external magnet in proximity to the cartridge and the aggregated PMPs are transported from the aqueous phase comprising the lysis buffer to air phase, followed by transporting them to the third chamber comprising a wash buffer (e.g., alcohol-methanol or ethanol) (Step 2). In step 3, the aggregated PMPs are resuspended by removing the magnet and optionally agitating the cartridge. The PMPs are once again captured using a magnet and transported to the chamber comprising elution buffer.

A sample processed by the methods, cartridge, and systems provided herein may be a biological sample, e.g., the sample may be a sample of whole blood, serum, plasma, sputum, nasal fluid, saliva, mucus, semen, vaginal fluid, a tissue, urine, organ, and/or the like of a mammal (e.g., a human, a rodent (e.g., a mouse), or any other mammal of interest). In other aspects, the sample is a collection of cells from a source other than a mammal, such as bacteria, yeast, insects (e.g., drosophila), amphibians (e.g., frogs (e.g., Xenopus)), viruses, plants, or any other non-mammalian nucleic acid sample source.

### Sample Preparation Cartridges

The sample preparation cartridges used to carry out the methods disclosed herein may include a plurality of sample processing regions or chambers comprising reagents for biological molecule or cell purification, modification, analysis, and/or detection; and a gaseous phase (air) in between (e.g., separating) two or more of the regions/chambers.

In some embodiments, there are three chambers. In some embodiments, there are four chambers. In some embodiments, there are five chambers. In some embodiments, there are six or more chambers (e.g., 7, 8, 9, 10, 11, and up to 30 chambers). In some embodiments, the air provides a contiguous barrier between two or more of the chambers (i.e., a sample passes from an aqueous phase into air and directly out of the air into the next aqueous phase).

Exemplary devices that can be used to prepare a sample according to the method of the present disclosure are depicted in FIGS. 1A-1G of U.S. Pat. No. 9,766,166, FIGS. 1-5 of U.S. Pat. No. 10,040,062, and FIG. 2. of U.S. Pat. No. 8,304,188. The disclosures of these devices are herein incorporated by reference. Other devices that may be used for performing the method for sample processing are depicted schematically in FIGS. 1A-1B and shown in FIGS. 1C-1D.

In certain examples, the method may be performed using a sample preparation cartridge. The sample preparation cartridge may have a substantially cylindrical shape. For example, the sample preparation cartridges include a cylindrical structure including a top end, a bottom end and an annular wall extending between the top and bottom ends. The cylindrical structure includes a plurality of chambers located in the annular wall, where the chambers extend between an exterior surface of the annular wall and an interior of the cylindrical structure, where the annular wall comprises cavities forming an open side of each of the chambers; and one or more channels providing fluidic communication between the plurality of chambers, where the channels are formed by recesses in the annular wall and comprise an open side; and one or more covers affixed over exterior surface of the annular wall to cover and fluidically seal the open side of the chambers and the open side of the recesses.

Additionally, in certain embodiments, the sample preparation cartridge may also include: a buffer pack, a sealing lid assembly, a protective cover, and a cap. The cartridge may also include a sample input component. The sample preparation cartridge is configured for use with a cylinder housing comprising a magnet.

By cylindrical, it is meant that the cylindrical structure may be substantially a right circular cylinder. The cylindrical structure may be rotatable around the axis formed by a line connecting the center of the bottom end of the cylindrical structure with the center of the top end of the cylindrical structure. For example, the cylindrical structure may rotate clockwise when the cylindrical structure is viewed from above looking down onto the top of the cylindrical structure or may rotate counterclockwise. Alternatively, the cylindrical structure may rotate both clockwise and counterclockwise. Rotation of the cylindrical structure may be used for mixing contents of the one or more chambers or positioning a magnet present in the cylinder housing adjacent a chamber to cause aggregation of magnetic particles present in the chamber and/or to transfer aggregated magnetic beads from one chamber to another, etc.

As summarized above, the cylindrical structure comprises a plurality of cavities in the annular wall that form a plurality of open-sided chambers on the annular wall. For example, the plurality of cavities may be indentations in the annular wall that deform the continuous surface of the annular wall. By open sided, it is meant that the annular wall does not cover such side of the chamber. In certain instances, the deformed annular wall may form closed sides of the chambers, and the area corresponding to the side of the annular wall that was deformed to form the cavity may form the open side of the chambers.

According to certain embodiments, the open sides of the plurality of chambers are located on the exterior of the annular wall. For example, the annular wall may be deformed inward from the outside to form an inwardly deformed cavity in the annular wall. In such case, the open side of the chamber may be the area corresponding to the side of the annular wall that was deformed inward to form the cavity. In such instances, the annular wall that has been inwardly deformed may form closed sides of the chambers. The volume of a chamber may represent a measurement corresponding to the volume of the indentation in the annular wall. The chambers may be any convenient volume, and in some instances may vary from 1 cm 3 to about 5 cm 3, such as 1 cm 3 to 3 cm 3 or 2 cm 3 to 5 cm 3. In other instances, the chambers can contain any convenient volume of fluid, and in some instances may vary from 1 μL to about 5,000 μL, such as 1 μL to 100 μL or 1,000 μL to 3,000 μL or 2,000 μL to 5,000 μL. Each chamber of the plurality of chambers may have the same volume or may have different volumes. The depth of the chamber, measured as the distance from the outside surface of the annular wall to the inner side of the chamber, may be any convenient size, and in some instances, may be 0.1 cm or greater, such as 1 cm or 5 cm. Each chamber of the plurality of chambers may have the same depth or may have different depths.

According to certain embodiments, the plurality of chambers is positioned proximal to each other on the annular wall. For example, the distance between a lateral border of a first chamber and the closest lateral border of a second chamber may be about 0.1 cm or more, such as 0.5 cm to 1 cm, e.g., 0.5 cm or 0.75 cm or 5 cm. The distances between lateral sides of pairs of chambers positioned next to each other may be the same for the plurality of chambers or may differ.

As summarized above, sample preparation cartridges include one or more channels that provide fluidic communication between the plurality of chambers. In certain aspects, the channels are wide enough that one or more PMPs can be transported therethrough. In certain embodiments, one or more of the channels between chambers are formed by a recess in the annular wall. By recess in the annular wall, it is meant an indentation or a cavity in the annular wall capable of providing fluidic communication between chambers. In some cases, the recess is formed in the outside surface of the annular wall, such that a first chamber and a second chamber that are formed with open sides on the exterior surface of the annular wall are interconnected by a recess in the outside surface of the annular wall between such first chamber and second chamber. The recesses in the annular wall may be any convenient length, width and depth. In certain examples, the height of the recesses can range from 0.5 mm to 5 mm, e.g., 2.5 mm, the depth can be 0.2 mm to 1 mm, e.g., 0.5 mm and the length can be 1 mm to 10 cm, e.g., 4-5 cm.

In certain embodiments, the recesses are positioned on the lateral sides of the plurality of chambers. By lateral sides of the plurality of chambers, it is meant the left- or right-hand sides and not the top or the bottom sides of the chambers, when the axis of the cylindrical structure formed between the center of the bottom end and the center of the top end of the cylindrical structure is oriented vertically. By positioning recesses on the lateral sides of the plurality of chambers, it is meant that a recess may interconnect the right-hand side of a first chamber with the left-hand side of a second chamber, such that such first and second chambers are in fluidic communication with each other via the recess. Recesses between chambers may be substantially straight lines between a point on a first chamber opposite to a point on a second chamber such that the recesses are substantially parallel to a plane defined by the bottom end of the cylindrical structure. A recess between a first and second chamber may have substantially the same width and depth in the annular wall across the entire length of the recess or may vary. Recesses between different pairs of chambers may have different dimensions or same dimensions. Recesses may be shaped as convenient such to allow PMPs may be translated therethrough.

In certain embodiments, the recesses are positioned on the lateral sides of one or more chambers at a substantially constant height above the bottom end of the cylindrical structure. In these embodiments, the recesses between pairs of chambers may be substantially linear. In these embodiments, the recesses and the chambers may be shaped such that a path exists starting from the leftmost position on the leftmost chamber through each of the plurality of chambers to the rightmost position of the rightmost chamber, in a straight line. The recesses on the lateral sides of one or more chambers may be positioned at any convenient height above the bottom end of the cylindrical structure. In certain of these embodiments, the height above the bottom end of the cylindrical structure at which the recesses are positioned corresponds to the vertical midpoint of one or more of the chambers.

In certain embodiments, the shape of one or more of the plurality of chambers is generally rectangular. By generally rectangular chamber, it is meant that the two-dimensional shape of the indentation into the annular wall is longer than it is wider. The height and width of each chamber may be any convenient height and width. The height and width of each rectangular chamber may be identical or may differ.

In certain embodiments, the shape of a chamber connected to another chamber by one or more channels is such that with respect to a lateral portion of the chamber that is proximal to a channel, the height of the chamber at each lateral position of the chamber decreases the closer such position is to the channel. In some cases, the height of such chamber at each lateral position decreases linearly so as to form a tapered region. Such a tapered entrance to the recess may facilitate transport of aggregated PMPs from the chambers to the channel.

In certain embodiments, one or more of the chambers comprises a drain hole. By drain hole, it is meant an opening through which fluid may exit the chamber. For example, fluid may drain from a drain hole located at the bottom of a chamber under the influence of the force of gravity. Alternatively, fluid may be plunged out of the chamber upon the application of pressure to fluid in the chamber by a plunger.

The one or more of the chambers may include an opening which is configured for venting of the chamber, filling of the chamber with a fluid, and/or draining of fluid from the chamber.

In certain embodiments, the interior of the cylindrical structure comprises one or more wells. By wells, it is meant one or more enclosures within the inside of the cylindrical structure. The enclosures may be any convenient size or shape. For example, the enclosures may be substantially cylindrical, with a closed bottom end, an annular wall, and an open top end. In these embodiments, cylindrical structures may further comprise channels in the cylindrical structure that provide fluidic communication between such wells and one or more of the plurality of chambers. In some instances, each well is interconnected with a distinct chamber via one or more channels.

In certain embodiments, the plurality of chambers forms a first chamber, a second chamber and a third chamber. In certain embodiments, the first chamber is adjacent to the second chamber; the second chamber is adjacent to the first and third chambers; and the third chamber is adjacent to the second chamber. In certain embodiments, the cylindrical structure further includes a first recess in the annular wall providing fluidic communication between the first and second chambers, and a second recess in the annular wall providing fluidic communication between the second and third chambers. In certain embodiments, the first chamber is a lysis chamber; the second chamber is an immiscible phase chamber, i.e., air; and the third chamber is an elution chamber. By lysis chamber, it is meant a chamber that during use of the sample preparation cartridge contains lysis buffer, such as a fluid that is a lysis buffer. By immiscible phase chamber, it is meant a chamber that during use of the sample preparation cartridge contains an immiscible phase, such as a fluid that is immiscible with aqueous phase. In some cases, the immiscible phase can be oil for example, where the PMPs are transported from air chamber to wash solution and then to an immiscible phase chamber (oil or air) and finally to elution chamber. By elution chamber, it is meant a chamber that during use of the sample preparation cartridge contains a fluid into which an analyte bound to the PMPs may be released. In certain embodiments, the fluid may be referred to as s an elution buffer. In certain embodiments, the elution buffer may be compatible with subsequent downstream processing of the isolated analyte. For example, the elution buffer may be an amplification buffer. The amplification buffer may be suitable for performing amplification of the isolated analyte, by, e.g., isothermal amplification or PCR.

The first chamber may include an opening at the top of the chamber. This opening may be configured as an inlet. The inlet may be configured for introducing a lysis buffer or lysing agent, a sample, and/or a mixture thereof. Thus, the inlet may have a diameter compatible for pipetting, injecting, or pumping a lysis buffer, a sample, and/or a mixture thereof. In some cases, the second chamber may also include an opening at the top of the chamber. This opening may be configured as an inlet for introducing an immiscible phase, e.g., oil into the second chamber. In some cases, the third chamber may also include an opening at the top of the chamber. This opening may be configured as an inlet for introducing an elution buffer into the third chamber.

In certain examples, the first chamber may include a compartment positioned on the bottom region or underneath the bottom region of the first chamber. The compartment may include an opening fluidically connecting the compartment to the interior of the first chamber. The compartment may include the first population of magnetic particles and the second population of magnetic particles as described in the section on Method of Sample Preparation. The magnetic particles may be mixed together and then dried, e.g., lyophilized. In certain embodiments, the magnetic particles may be mixed together, placed in the compartment, and subsequently dried to provide a lyophilized preparation. In certain embodiments, the magnetic particles may be mixed together, dried, and subsequently placed in the compartment to provide a lyophilized preparation. In certain embodiments, the first chamber includes an opening at the bottom of the chamber, wherein the opening is configured as an inlet for lysis buffer and wherein the first chamber comprises an opening at the top of the first chamber configured as a sample inlet. In certain embodiments, the compartment includes an inlet fluidically connecting the compartment to a channel and an outlet fluidically connecting the compartment to the interior of the first chamber.

In certain examples, the second chamber may not include an opening other than the interconnections with the first and third chambers. The second chamber may contain air. When the first and third chambers are filled with a liquid the air in the second chamber is compressed due to lack of a vent in the second chamber. As noted herein, the compressed air serves as a “wash” environment for PMPs transferred from the first chamber to the third chamber via the second chamber comprising the compressed air.

In certain examples, the third chamber includes an opening at a bottom region of the chamber. The opening is configured for filling the third chamber. The t opening is distinct from a drain hole present at the bottom region of the chamber. In certain cases, the drain hole may have a smaller diameter than the opening configured for filling the third chamber such that the drain hole does not allow liquid to pass through under atmospheric pressure and requires a higher pressure to allow passage of liquid. In some cases, the drain hole at the bottom of the third chamber is fluidically connected to one or more collection containers. The collection containers may be two separate tubes, e.g., thin wall polypropylene tube suitable for PCR or similar thin-walled containers or strips conducive to thermal cycling reactions. drain hole at the bottom of the third chamber may be fluidically connected to two channels that split from the drain hole to fill the two collection containers with substantially equal volume of liquid drained from the third chamber.

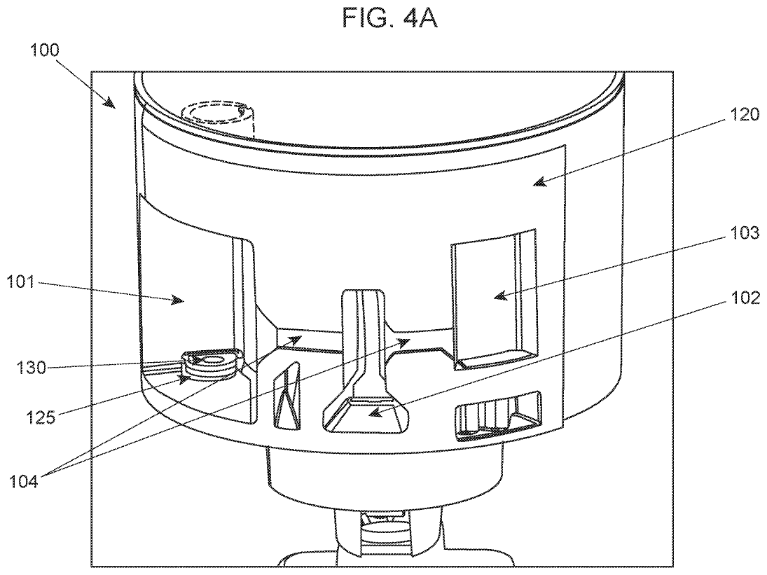

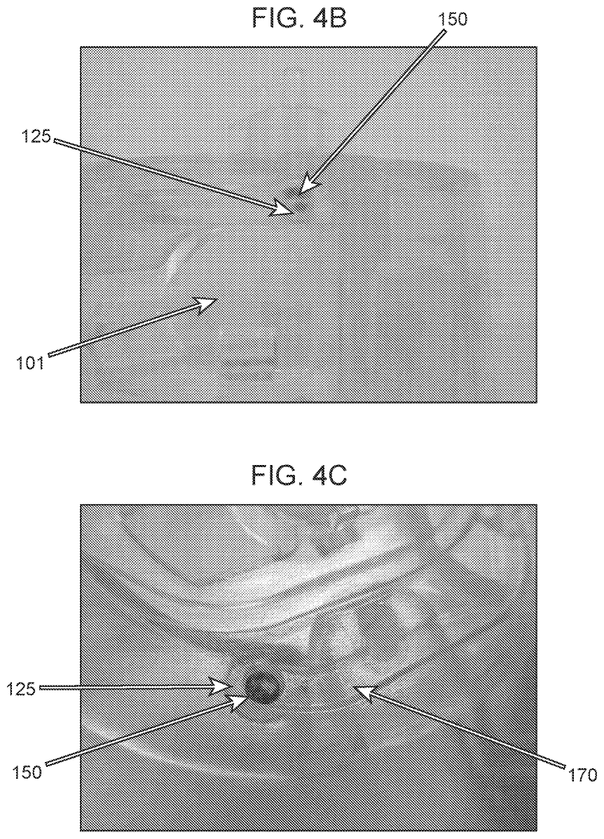

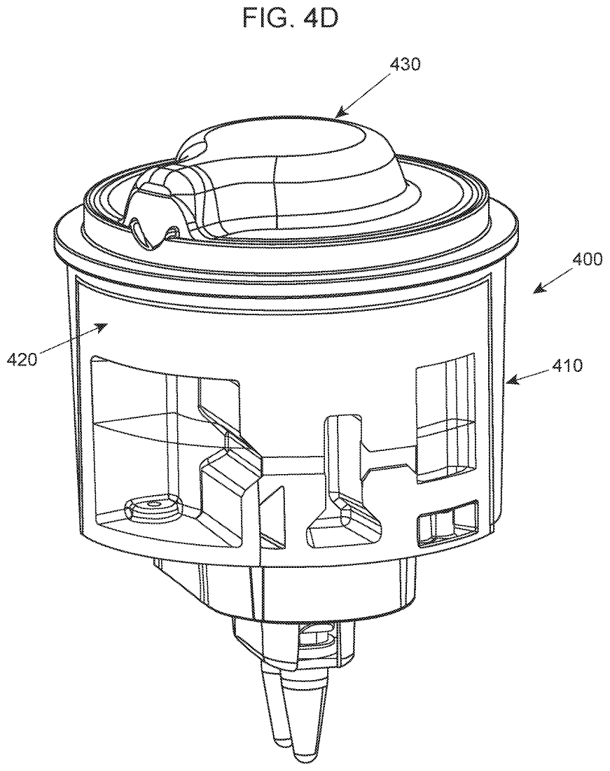

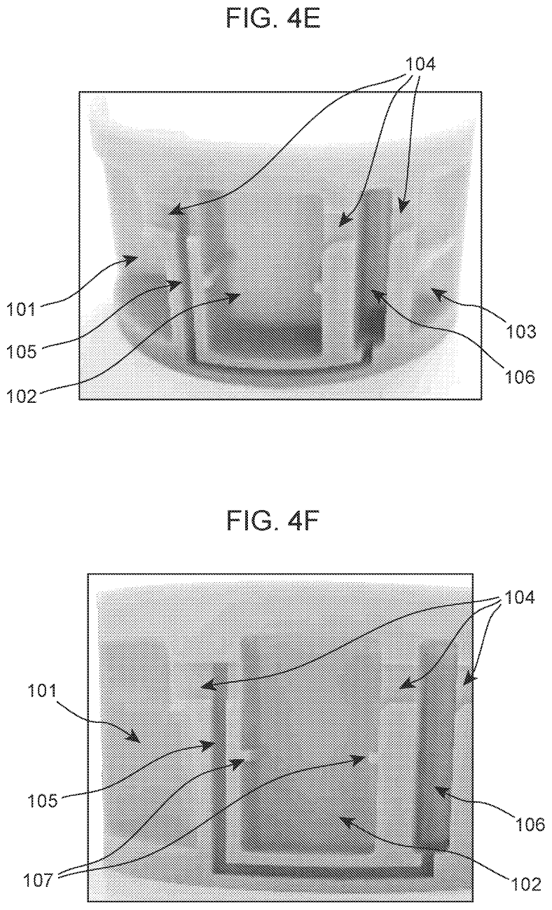

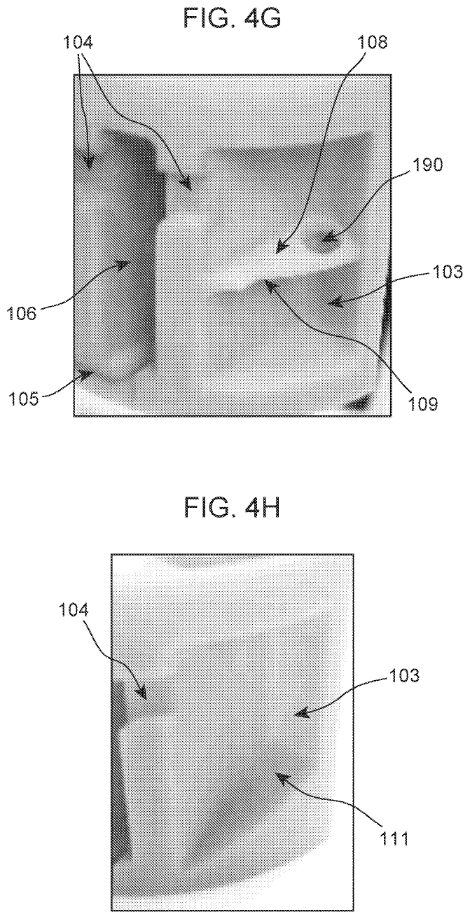

A cylindrical cartridge according to one embodiment is shown in FIG. 4A. In this example, the cylindrical cartridge 100 includes three cavities in the annular wall that form three open-sided chambers 101, 102, 103 on the annular wall and two recesses that form open-sided interconnections 104. As seen, the open sides of the chambers 101, 102, 103 are located on the exterior of the annular wall, and the chambers 101, 102, 103 are positioned adjacent to each other. The two interconnections 104 between the chambers 101, 102, 103 provide fluidic communication between the chambers. In this example, the interconnections 104 are channels that are recesses in the annular wall, and the interconnections 104 are positioned on the lateral sides of the plurality of chambers 101, 102, 103. As illustrated in the figure, the recesses that form interconnections 104 between the chambers 101, 102, 103 are at a substantially constant height above the bottom end of the cylindrical structure 100. Also illustrated in FIG. 4A is a compartment 125 located in the bottom region of the first chamber. The compartment includes a dried mixture of the first and second populations of magnetic particles disclosed herein. The compartment includes an opening 130 through which the magnetic particles can enter the first chamber. The compartment is fluidically connected to a channel connected to a buffer pack that supplies an aqueous phase such as a lysis buffer to the first chamber via the compartment. FIG. 4B shows close up image of a cartridge for sample preparation which image shows the cartridge in an upside-down orientation. The first chamber 101 is visible. The compartment 125 is also visible. PMPs 150 are disposed in the compartment 125. FIG. 4C shows a picture of the bottom region of sample preparation cartridge viewed from below. A film that covers the compartment 125 is removed to aid visualization of PMPs 150. Also visible is a channel that leads to the bottom region of the compartment 125 and can be used to supply an aqueous phase (e.g., lysis buffer) to the compartment.

The sample preparation cartridges include one or more covers that closes the open sides of the plurality of chambers and the interconnections to form channels. In certain aspects, a cover curves to mate with the outside surface of the cylindrical structure. When the cover closes a chamber, a fluid disposed in the chamber is fully contained in the chamber. Use of a cover to form a wall of the chambers in the cylindrical cartridge allows for a wall that is significantly thinner than the annular wall of the cylindrical structure. Use of a cover to form a wall of the chambers in the cylindrical cartridge allows for a wall that is made from a material different from the material of the cylindrical structure.

A cover may be made from any suitable material that can be curved and attached to the exterior surface of the annular wall. For example, the cover may be made from plastic (e.g., thermoplastic, such as, Cyclo Olefin Polymer or Cyclo Olefin Copolymer), metal, paper, glass, and the like. If metal material is used for the cover, the metal may be non-magnetic, i.e., not include substantial amount of iron. A paper cover may include a non-wettable coating, e.g., a wax coating. The cover may be substantially opaque or substantially transparent. The cover may be attached to the annular wall by any suitable means such as via an adhesive, locally heating the exterior of the annular wall or the cover or both, by snapping the cover into a groove(s) created in the annular wall, by screwing the cover into the annular wall, and the like. The cover may be sufficiently thin so as to not significantly decrease in the chambers the magnetic force of the external magnet. For example, the cover may be sufficiently thin to allow paramagnetic particles (PMPs) present in a chamber to be aggregated in response to the external magnet being located adjacent the chamber and to allow the aggregated PMPs to traverse thorough a channel connecting adjacent chambers in response to relative movement of the cylindrical structure and the external magnet. The cover may have a thickness of less than 1 cm, less than 0.5 cm, less than 0.1 cm, e.g., 1 mm-5 mm, or 0.1 mm-5 mm, or 0.1 mm-1 mm, or 0.1 mm-0.5 mm. In certain embodiments, the cover may be a film, e.g., an adhesive film.

According to certain embodiments, the cover fluidically seals the open sides of the plurality of chambers. By fluidically seals the open sides of the plurality of chambers, it is meant that when the cover is positioned on the cylindrical structure, the space inside the chambers is not in fluidic communication with the space outside the cylindrical structure, via the open side of the chambers.

According to certain embodiments, the interior surface of the cover facilitates movement of the magnetic particles thereon. By facilitating movement of PMPs, it is meant that the interior surface of the cover may be configured such that PMPs may be more reliably translated from a first position on the cover to a second position on the cover while remaining in contact with the interior surface of the cover. For example, the interior surface of the cover may be polished to reduce friction between PMPs and the interior surface of the cover as the PMPs move along the cover. By translated from a first position on the cover to a second position on the cover, in certain cases, it is meant that the PMPs are moved along the interior of the cover.

In certain embodiments, sample preparation cartridges may include a buffer pack. A buffer pack may comprise one or more fluid packs. Each fluid pack may contain a fluid. In some embodiments, fluid packs may comprise a lysis buffer pack and an elution buffer pack. In other embodiments, fluid packs may comprise each of a lysis buffer pack, an immiscible phase pack and an elution buffer pack. In certain embodiments, the immiscible phase may comprise an oil. In certain embodiments, the immiscible phase may comprise air. In some instances, one or more of the fluid packs may further comprise PMPs. The fluid pack may contain any convenient amount of PMPs, measured based on, for example, the volume or the weight of PMPs. For example, the PMPs may be mixed with a fluid when included in a fluid pack. In some instances, PMPs may be included in a fluid pack that comprises a lysis buffer.

In certain embodiments, the buffer pack is configured to fit within the wells of the cylindrical structure. For example, when the wells are shaped as substantially hollow cylinders, the buffer pack may be shaped as cylinders that fit within the wells of the cylindrical structure. Buffer packs are described in greater detailed in an U.S. Provisional Patent Application titled “Magnetic Particle Separation Device Buffer Pack and Cap Design,” Attorney Docket No. ADDV-082PRV, co-filed with this application, which application is herein incorporated by reference in its entirety.

In some embodiments, the lysis buffer can be formulated to release nucleic acid from a broad spectrum of samples, such as tissue samples, cells, viruses, or body fluid samples. The lysis buffer can also be designed to lyse all types of pathogens, such as viruses, bacteria, fungi, and protozoan pathogens. Such lysis buffer can contain a chaotropic agent, particularly, guanidine hydrochloride. In addition, the lysis buffer may include other reagents, such as, surfactants, anti-foam chemicals, buffers, etc.

In certain embodiments, the sample preparation cartridge includes a sealing lid assembly. Sealing lid assembly includes sealing plate positioned on top end of the cylindrical structure and a protective cover positioned over the sealing plate. The protective cover may enclose the periphery of the sealing plate and snap on and around the top region of the cylindrical structure to keep the sealing plate in place.

The sealing plate fits on the top end of the cylindrical structure and closes the top end. In certain embodiments, the sealing plate may include opening aligned with an opening in the third (elution) chamber for removing elution buffer for analysis of eluted nucleic acid. In other embodiments, the sealing plate may further include a plunger assembly. The plunger assembly may include a gasket seal mounted on a shaft, a spring, and a trigger that engages the spring and the shaft. The shaft may be any convenient length, such as a length that is less than or equal to the height of a corresponding chamber. The gasket seal may be shaped so that the size of the operative end of the gasket seal is substantially similar to the corresponding chamber with which the plunger is integrated. In these embodiments, the spring may apply tension to the plunger in a retracted position. That is, when the plunger is retracted, the spring is under tension. By retracted, it is meant that the gasket seal end of the plunger is retracted. When in the retracted position, the plunger may not plunge fluid from a corresponding chamber. The amount of tension applied by the spring when the plunger is retracted corresponds to the amount of tension applied by the spring to the plunger when the plunger is no longer retracted and may vary as desired. By a trigger that engages the spring and the shaft, it is meant that the trigger may control the release of a spring under tension holding the plunger in a retracted position.

In certain embodiments, the trigger and the spring are mechanically interlocked so that the trigger is armed when the plunger is in the retracted position. By armed, it is meant that depressing the trigger releases tension on the spring, thereby causing the plunger to move from the retracted position to the plunged position.

In these embodiments, the gasket seal of the plunger may be positioned to engage with one of the chambers. By engaging with one of the chambers, it is meant that the plunger assembly is positioned so that when the plunger assembly is in the plunged position, the gasket seal of the plunger nearly fills the bottom portion of the chamber, and when the plunger assembly is in the retracted position, the gasket seal of the plunger does not fill the bottom portion of the chamber. That is, the movement of the plunger from a retracted to a plunged position is such that the plunger may plunge the chamber. By plunging the chamber, it is meant that as the plunger transitions from a retracted to a plunged position, the gasket seal of the plunger engages with the chamber to apply pressure to any fluid in the chamber.

In these embodiments, the trigger may be positioned on the sealing lid assembly so that the trigger protrudes a distance beyond the outside wall of the cylindrical structure. By protruding a distance beyond the outside wall of the cylindrical structure, it is meant that the distance between the axis of the cylindrical structure and the furthest point on the trigger is greater than the distance between the axis of the cylindrical structure and the outside edge of the annular wall. The trigger may protrude any convenient distance beyond the outside edge of the annular wall. In these embodiments, the trigger may be oriented to be depressed in a lateral direction. By depressing the trigger, it is meant activating the trigger to release tension on the spring, to which the trigger is mechanically interlocked. By oriented to be depressed in a lateral direction, it is meant that the trigger is positioned so that, in order to depress the trigger, the trigger must be moved in a substantially lateral direction.

As summarized above, in certain embodiments, the sample preparation cartridge further includes a cap slidably positioned on the top of the cylindrical structure. By slidably positioned, it is meant that the cap can be positioned on the top of the cylindrical structure in such a manner that it can slide towards the cylindrical structure.

In certain embodiments, caps may comprise one or more arms positioned to mechanically engage the buffer pack. For example, the cap may be shaped substantially flat where one or more arms are attached to one flat side of the cap. Such arms may be any convenient size or shape. For example, the length of the arms may be long enough so that when the cap is positioned on top of the cylindrical structure, the arms can reach wells on the inside of the cylindrical structure.

In certain embodiments, the cap may include a plunger positioned such that when the cap is slid into the cylindrical structure the plunger enters a sample chamber and expels the sample to the lysis chamber. The sample chamber may be adjacent the first (lysis) chamber and connected to the lysis chamber via a channel. One of the arms of the cap may enter the lysis buffer pack forcing the lysis buffer out of the pack and into the first (lysis) chamber. Another arm of the cap may enter the immiscible phase pack, if present, and push the oil into the second (immiscible phase) chamber while a third arm of the cap enters the elution buffer pack and pushes the elution buffer into the third (elution) chamber.

A cartridge may be loaded into an instrument fitted with a magnet where the magnet is positioned with respect to the cartridge such that it can be used to move PMPs from the lysis chamber through the immiscible phase chamber into the elution chamber. The instrument may include a motor that engages the cartridge to rotate the cartridge relative to the magnet or the magnet may be configured to move along the annular surface of the cartridge.

A sample preparation cartridge according to one embodiment of the present disclosure is shown in FIG. 4D. In this example, the sample preparation cartridge 400 includes a cylindrical structure 410, a cover 420, a cap 430.

A sample preparation cartridge comprising certain modifications relative to the sample preparation cartridge depicted in FIGS. 4A-4D is shown in FIG. 4E-4G. FIG. 4E shows an air-break pathway 105 present between chambers 101 and 102 and an air-chamber 106 positioned between chambers 102 and 103. The air-break pathway 105, in this example, is connected to the air-gap chamber 106. In other examples, the air-break pathway 105 may not be connected to the air-gap chamber 106. For example, the air-break pathway may terminate at the bottom region of the cartridge. In certain embodiments, the bottom region of the air-gap chamber may be closed. The width of the air-gap is shorter than that of air-chamber or vice versa. In other examples, the width of the air-gap and air-chamber may be the same. In certain examples, baffles 107 may be introduced in chamber 102 and/or 101. FIG. 4F depicts chamber 102 with baffles 107 which may prevent liquid from splashing into channels 104 during mixing of the magnetic beads by, e.g., a back-and-forth rotation of the cartridge. Baffles 107 may be positioned at a position below the channels 104, e.g., 2 mm-10 mm, 3 mm-8 mm, or 5 mm-7 mm below the channels 104. The baffles may have a width of up to 5 mm, e.g., 1 mm-3 mm, as measured from the side wall of the chamber from which the baffle extends.

FIG. 4G shows chamber 103 with a shelf-baffle 108 extending transversely through the chamber. The shelf-baffle 108 includes a notch 109 and an opening 190 to allow passage of magnetic beads to an area below the shelf-baffle. The shelf-baffle may be used to prevent splashing of liquid during mixing of liquid present in chambers 102 and/or 103.

FIG. 4H shows a modified chamber 103 with a bottom wall 111 raised at one end such that the bottom wall closer to channel 104 forms an acute angle with a side wall of the chamber 103. This configuration may be an alternative to using a shelf-baffle to prevent splashing of liquid present in the chamber 103 into channel 104.

### Sample Preparation Systems

A sample preparation system comprising the sample processing cartridge described herein and a magnet operably placed in association with the cartridge such that the magnet can exert a magnetic force on the magnetic particles in the cartridge is provided. Exemplary sample preparation systems include a cylinder housing in which a cylindrical sample preparation cartridge can be removably disposed. By removably disposed, it is meant that the cylindrical cartridge can be fit into the cylinder housing in such a manner that the cylindrical cartridge can nonetheless be separated from the cylinder housing. For example, a user may dispose the cylindrical cartridge in the cylinder housing and may remove the cylindrical cartridge from the cylinder housing after sample preparation. As summarized above, the cylinder housing includes a magnet. By magnet, it is meant any object having the ability to produce a magnetic field external to itself. For example, the magnet may produce a magnetic field capable of attracting the magnetic particles. In some instances, the magnet may be a permanent magnet or an electromagnet. As used herein, “magnet” refers to a material or an article that may spontaneously or actively generate magnetic fields, where the strength of the magnetic fields can be measured by a conventional gaussmeter. A magnet can be a permanent magnet or an electromagnet. As used herein, “permanent magnet” refers to any object that is magnetized and creates its own persistent magnetic field. Suitable ferromagnetic materials for a permanent magnet include iron, nickel, cobalt, rare earth metals and their alloys. The term “permanent” does not mean such a magnet could not lose its magnetism, for example, through exposure to heat, physical shock, or an opposing magnetic field. In some examples, the permanent magnet comprises samarium cobalt (SmCo) alloy, aluminum nickel cobalt alloy (AlNiCo), neodymium iron boron (NdFeB) alloy, Nd₂Fe₁₄B, or ferrite. As used herein, “electromagnet” refers to any device that is capable of creating a magnetic field through the application of electrical energy. Electromagnets can include a core and a coil or other element for carrying current to generate magnetic fields.

In certain embodiments, the magnets are positioned proximal to the exterior of the annular wall of the cylindrical cartridge. In some embodiments, the magnet is external to the cylindrical cartridge and is used to transfer the magnetic particles between chambers of the cylindrical cartridge.

In certain embodiments, the cylindrical cartridge rotates within the cylinder housing. By rotate, it is meant that the cylinder housing permits the cylindrical cartridge freedom to rotate, for example, around the axis of the cylindrical cartridge formed by connecting the center of the top end with the center of the bottom end of the cylindrical structure. In other embodiments, the cylindrical cartridge maintains a fixed position in space and the cylinder housing rotates around the cylindrical cartridge.

In certain embodiments, a reusable magnet is used to process samples using a disposable consumable cylinder cartridge in a sample processing instrument. The use of the reusable magnet would reduce the waste with each consumable. In certain embodiments, the cylindrical housing is reusable.

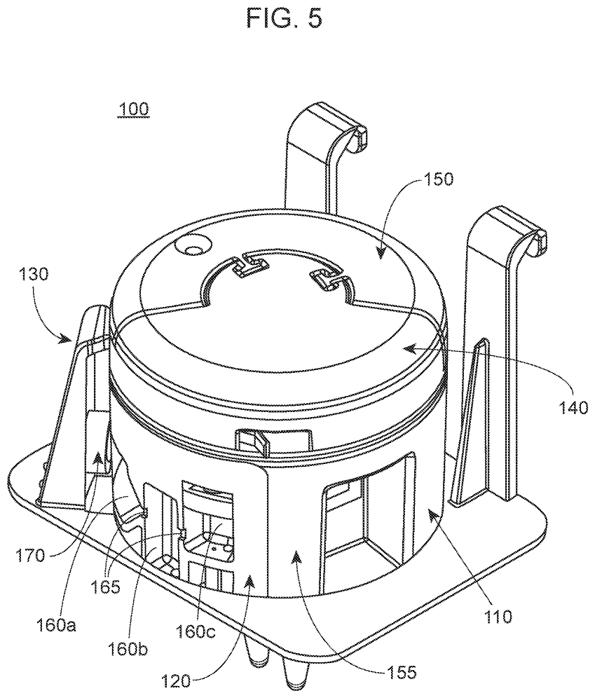

A sample preparation system that includes a sample preparation cartridge 100 and a cylinder housing 130, according to one embodiment of the present disclosure is shown in FIG. 5. In this example, the sample preparation cartridge 100 includes a cylindrical structure 110, a cover 120, a protective cover 140, and a cap 150. Also depicted in the figure are the annular wall 155 of the cylindrical structure and three cavities in the annular wall that form three open-sided chambers 160a-160c on the annular wall. As seen in the figure, the open side of each chamber 160a-160c is oriented towards the outside of the cylindrical structure 110. In addition, the open sides of the chambers 160a-160c are enclosed by the cover 120. In FIG. 5, cover 120 is depicted as transparent to aid visualization of the chambers 160a-160c but cover 120 need not be transparent. The cover 120 curves to mate with the outside surface of the annular wall 155, and fluidically seals the open sides of the chambers. Also seen in this figure are interconnections 165 between the chambers. As seen in the figure, the interconnections 165 are channels that are recesses in the annular wall between chambers. The figure also depicts the magnet 170 in the cylinder housing 130. As seen, the magnet 170 is positioned proximal to the exterior of the annular wall 155 of the cylindrical structure 110.

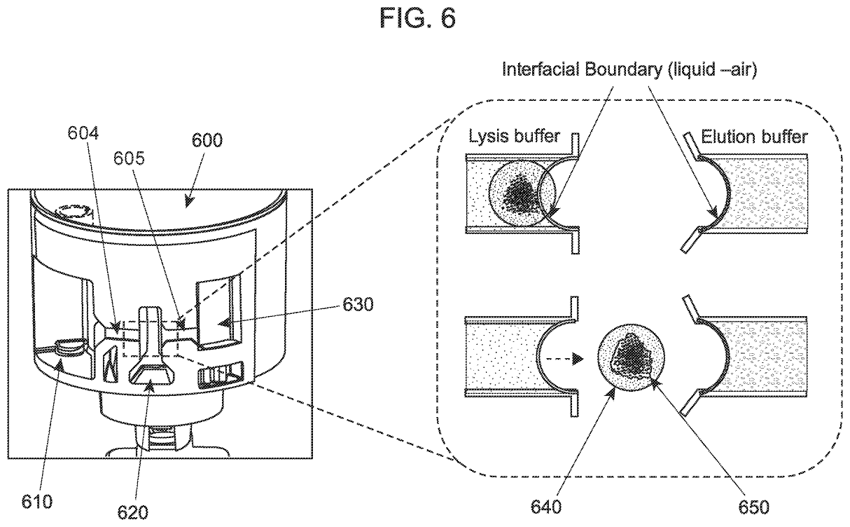

FIG. 6 shows an illustration of interfacial boundary between the air phase in the middle chamber and the aqueous phases in two chambers adjacent the middle chamber in a cylindrical sample preparation cartridge disclosed herein. While the illustration uses a sample preparation cartridge that is cylindrical, it is understood that the depictions also apply to other sample preparation cartridges that include an air phase and an aqueous phase, such as, linear sample preparation cartridges, e.g., see FIG. 1C. FIG. 6 shows a sample preparation cartridge 600 with an air chamber 620 flanked on both sides with aqueous chambers 610 and 630. The boxed region includes part of the first channel 604, the air chamber 620, and the second channel 605. During mixing of the PMPs with an aqueous phase, the first 604 and second 605 channels may become partially filled with aqueous phases. For example, lysis buffer present in the aqueous chamber 610 may spill into the first channel 604 during agitation of the sample preparation cartridge. An elution buffer present in the aqueous chamber 630 may spill into the second channel 605 during agitation of the sample preparation cartridge. The presence of air in the middle chamber 620 results in formation of an interfacial boundary at the interface between aqueous phase and air phase. The interfacial boundary substantially prevents the aqueous phases from flowing into the air chamber. In some examples, the air chamber includes a reservoir area to contain any aqueous phase that may spill into the air chamber, e.g., during agitation of the cartridge to mix the first and second populations of magnetic particles with the sample. A magnet 640 is depicted which is placed externally to the sample preparation cartridge. The magnet attracts and holds the magnetic particles and transports them across liquid-air interface, which results in a significant removal of liquid associated (e.g., bound to and/or trapped between the magnetic particles) with the magnetic particles. The magnet then transports the magnetic particles back to an aqueous phase (e.g., elution buffer).

### Automation for the Methods of Using the Sample Preparation Cartridges

Certain embodiments also provide sample preparation cartridges that can be actuated using a motor. The motor can be automated thereby automating the methods of using the sample preparation cartridges disclosed herein. The motor can also be controlled by a computer program, which when executed by a processor, causes the motor to perform the methods of using the cartridge disclosed herein.

In certain embodiments, the motor rotates the cylindrical structure in the increments of 1.8° angle.

In some embodiments, the motor rotates the cylindrical structure to return it to a predetermined position, for example, where the magnet is positioned proximal to the lysis chamber, immiscible phase chamber, or elution chamber.

The motor can be configured to provide only a fraction of the full 360° rotation. For example, the motor can be configured to provide only between 60° and 120° rotation, preferably, between 80° and 110° rotation, even more preferably, between 90° and 100° rotation, and most preferably, about 90° rotation.

In some embodiments, the motor can further facilitate mixing of the contents of the sample preparation cartridge. Such mixing can be performed by motor mediated shaking of the sample preparation cartridge. Appropriate mixing can be provided by the control of start position, amplitude, and/or speed of shaking. Mixing may reduce sample preparation time and/or improve sample preparation by reducing non-specific binding and improving homogenous mixing.

In certain aspects, rotating the cylindrical cartridge from a first position to a second position comprises rotating the cylindrical cartridge so that the entire span of the lysis chamber is rotated across the magnet. That is, the cylindrical cartridge may be rotated such that the entire lateral span of the lysis chamber is exposed to the magnet.

Similarly, in certain aspects, rotating the cylindrical cartridge from a second position to a third position comprises rotating the cylindrical cartridge so that the entire span of the immiscible phase chamber is rotated across the magnet. That is, the cylindrical cartridge may be rotated such that the entire lateral span of the immiscible phase chamber is exposed to the magnet.

The methods of the present disclosure may include the additional steps of filling the lysis chamber with a lysis buffer and paramagnetic particles from a fluid pack housed within a buffer pack and filling an elution chamber with an elution buffer from a fluid pack housed within the buffer pack. In embodiments utilizing a non-air immiscible phase, the steps may additionally include filling an immiscible phase chamber with an immiscible phase from a fluid pack housed within the buffer pack.

In certain embodiments, fluid is transferred from a fluid pack housed within a buffer pack to a chamber by applying pressure to the fluid in the fluid pack to force the fluid through channels in the cylindrical structure of the sample preparation cartridge. For example, the fluid may comprise a lysis buffer, in some cases including paramagnetic particles, an immiscible phase and an elution buffer. In some cases, the immiscible phase comprises oil.

When fluid is transferred from a fluid pack, in certain embodiments, pressure is applied to the fluid in the fluid pack by applying mechanical force to a cap of the sample preparation cartridge that comprises arms to engage the fluid pack. By cap, it is meant any convenient mechanical structure with arms to engage the fluid pack. For example, the cap may comprise a substantially flat base from which arms protrude from one side such that when force is applied to the flat side of the cap, such force is transferred along the arms protruding from the base, which in turn engage fluid in the fluid pack thereby applying pressure to the fluid in the fluid pack and forcing it through channels in the cylindrical structure.

The methods of the present disclosure may include the additional step of transferring eluted nucleic acids out of the elution chamber of the sample preparation cartridge by plunging the contents of the elution chamber through a drain hole in the chamber. Plunging the elution chamber may take any convenient form. For example, the sample preparation cartridge may include a plunger assembly, including a plunger configured to engage the elution chamber that may be automatically triggered to plunge the elution chamber upon rotating the cylindrical structure to a specified position.

When the eluted nucleic acids are plunged out of the elution chamber, in certain embodiments, the sample preparation cartridge further comprises a plunger, a spring and a trigger interlocked together so that plunging the elution chamber comprises applying pressure to the trigger to release tension on the spring thereby driving the plunger into the elution chamber. In certain embodiments, the cylindrical structure is rotated to a fourth position to allow a mechanical arm to apply pressure to the trigger. In such cases, the trigger may protrude beyond the exterior radius of the cylindrical structure. By mechanical arm, it is meant any convenient device for use in depressing the trigger. For example, such a mechanical arm may be mounted in a fixed location and positioned to engage the trigger only when the cylindrical cartridge is rotated to a position where the mechanical arm abuts the trigger.

In certain embodiments, a sample comprising cells is introduced to the lysis buffer by applying pressure to a sample input component of the sample preparation cartridge and thereby introducing the sample comprising cells into the lysis buffer. By sample input component, it is meant any convenient structure for enclosing cells such that when a force is applied to the structure, pressure is applied to the sample thereby forcing the sample from the structure into the lysis chamber of the sample preparation cartridge.

### Example 1: Use of Air Phase for Sample Preparation

Air chambers generally exhibit higher interfacial energy and require much higher pull-through force (liquid-air penetrating force). The present invention provides a solution to the requirement of a higher-overall force for transporting PMPs from the aqueous phase to the air phase by adding “helper beads” to assist with this boundary transition. These helper beads are typically hydrophilic, larger in size and/or denser in magnetite, and have high magnetic response. Due to the size and larger mass per bead, they can become slightly magnetized in presence of a permanent magnet. This induced magnetism of the helper beads can assist and hasten the aggregation of smaller beads in solution surrounding it. While the PMPs are functionalized to capture a target analyte, the helper beads do not bind to the target analyte.

Approximately 800,000 magnetic beads having an average size of 2.7 um diameter (JSR Scientific MS300) were mixed with approximately 16,000 to 32,000 helper beads having an average size of 10 um diameter (Sigma 49664) in approximately 1:1 (50% bead mass) per reaction.

In a separate experiment, approximately 8.2 million magnetic beads having an average size of 3.7 um diameter (Qiagen MagAttract) were mixed with approximately 3,000 to 10,000 helper beads having an average size of 100 um diameter (GE Healthcare) in approximately 1:1 (50% bead mass) per reaction.

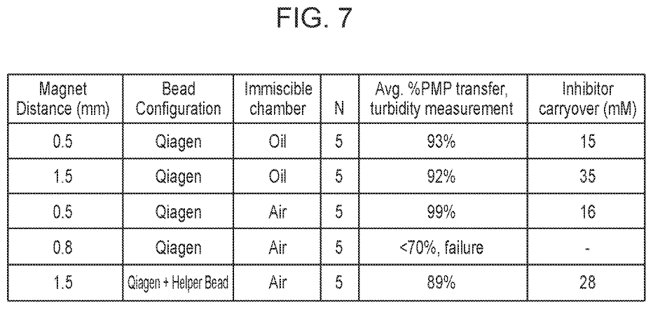

FIG. 7 shows the results from using air or an oil as the immiscible phase. Air-transfer required close placement of the external magnet. Transfer through oil is not as sensitive to magnet placement. When using Qiagen capture beads alone, the maximum separation distance between the magnet and the cartridge, e.g., the cartridge show in FIG. 1D, is 0.5 mm. Beyond this distance, a substantial amount of the beads are lost at the aqueous-air interface. An acceptance criterion of about 90% transfer is expected, since amount of PMPs transferred directly affects assay sensitivity and nucleic content retrieved. A 90% transfer yield is attainable by adding helper beads. Helper beads also allow for placement of the magnet up to 1.5 mm away from the cartridge. Turbidity of aqueous phase comprising the PMPs was used as a measurement for percent PMP transfer. Inhibitor carryover was used to compare effect of the immiscible phases (air or oil) on reducing transfer of aqueous phase with the PMPs.

Accordingly, the preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

What is claimed is:

1. A method for processing a sample comprising or suspected of comprising a target analyte, the method comprising:
contacting a sample with a first population of magnetic particles and a second population of magnetic particles in an aqueous phase in a first region of a sample processing cartridge,
wherein the first population of magnetic particles is capable of associating with the target analyte, and
wherein the magnetic particles in the second population are at least two-times larger in size than the magnetic particles in first population; and
transporting the first and second populations of magnetic particles from the aqueous phase in the first region to an air phase in a second region of the cartridge by applying a magnetic force to the magnetic particles.

2. The method of claim 1, wherein the magnetic particles in the first population have a diameter of 500 nm-10 um.

3. The method of claim 1 or 2, wherein the magnetic particles in the second population have a diameter that is 2×-20× the diameter of the magnetic particles in the first population.

4. The method of any one of claims 1-3, further comprising transporting the first and second populations of magnetic particles from the air phase in the second region to an aqueous phase in a third region of the cartridge by applying a magnetic force to the magnetic particles.

5. The method of claim 4, wherein applying the magnetic force causes the magnetic particles to aggregate in an area in the first region, which area is adjacent to the source of the magnetic force and wherein the transporting comprises maintaining the magnetic force on the aggregated magnetic particles, moving the aggregated magnetic particles to the air phase in the second region of the cartridge, and moving the aggregated magnetic particles to the aqueous phase in the third region of the cartridge.

6. The method of any one of claims 1-5, wherein transporting the first and second populations of magnetic particles comprises moving a magnet generating the magnetic force relative to the different regions of the cartridge.

7. The method of any one of claims 1-5, wherein transporting the first and second populations of magnetic particles comprises moving the cartridge or a portion thereof relative to a magnet generating the magnetic force.

8. The method of any one of claims 1-5, where the cartridge is substantially planar or substantially cylindrical.

9. The method of claim 8, wherein the cartridge is substantially planar and comprises a first plate placed in a spaced apart manner from a second plate, wherein the first and second plates are held in stationary position relative to each other.

10. The method of claim 8, wherein the cartridge is substantially planar and comprises a first plate placed in a spaced apart manner from a second plate, wherein the first and second plates are movable relative to each other such that the plates are held in a spaced apart and slidably movable configuration.

11. The method of any one of claims 8-10, wherein the cartridge is substantially planar and does not include separate chambers and the aqueous phase in the first region is an aqueous droplet and the air phase in the second region is air present between the first and second plates, and if present, the aqueous phase in the third region is an aqueous droplet.

12. The method of any one of claims 1 to 9, wherein the first region is a first chamber that comprises the aqueous phase and the second region is a second chamber that comprises the air phase.

13. The method of claim 12, wherein the first and second chambers are connected via a first channel, wherein a difference in pressure between the first and second chambers establishes a liquid-air interface in the first channel.

14. The method of any one of claims 4 to 9, wherein a third region is a third chamber comprising the aqueous phase.

15. The method of claim 14, wherein the first and second chambers are connected via a first channel, wherein a difference in pressure between the first and second chambers establishes a liquid-air interface in the first channel and wherein the second and third chambers are connected via a second channel, and a difference in pressure between the second and third chambers establishes an air-liquid interface in the second channel.

16. The method of claim 1, further comprising transporting the first and second populations of magnetic particles from the air phase in the second region to an aqueous phase in a separate cartridge, wherein the transporting comprises maintaining a magnetic force on the magnetic particles till the second region is in association with the aqueous phase in the separate cartridge and removing the magnetic force, thereby allowing the magnetic particles to be released into the aqueous phase.

17. The method of claim 16, wherein the first and second regions of the cartridge are removably associated.

18. The method of claim 16 or 17, wherein the first region is a first chamber, the second region is a transfer plate and the third region is a third chamber.

19. The method of any one of claims 1-18, wherein the contacting comprises contacting a lysis buffer comprising the first and second populations of magnetic particles with the sample.

20. The method of any one of claims 1-19, wherein the contacting comprises placing the sample in the first region followed by introducing into the first region the first and second populations of magnetic particles.

21. The method of any one of claims 1-20, wherein the target analyte comprises a cell, a virus, a protein, or a nucleic acid.

22. The method of claim 21, wherein the target analyte comprises a nucleic acid present in a cell or a virus.

23. The method of any one of claims 1-22, wherein the contacting results in disruption of a cell or virus present in the sample to release the nucleic acid present in the cell or virus, respectively.

24. The method of claim 23, wherein the second population of magnetic particles is incapable of associating with the target analyte, wherein optionally the target analyte comprises nucleic acids.

25. The method of any one of claims 12-24, wherein the second chamber comprises compressed air wherein the compressed air is generated by filling of the first and third chambers with aqueous solution at atmospheric pressure.

26. The method of any one of claims 12-25, wherein the aqueous phase in the third region comprises an elution buffer.

27. The method of any one of claims 1-26, wherein the aqueous phase in the third region comprises a wash solution and the cartridge comprises a fourth region comprising air or a immiscible substance and a fifth region comprising an elution buffer, wherein optionally, the fourth and fifth regions are chambers.

28. The method of claim 27, further comprising transporting the first and second populations of magnetic particles from the third region to the fifth region via the fourth region.

29. The method of any one of claims 1-28, wherein the transporting comprises moving a magnetic field relative to the cartridge, while the cartridge remains stationary.

30. The method of any one of claims 1-28, wherein the transporting comprises moving the cartridge relative to a stationary magnetic field.

31. The method of any one of claims 1-28, wherein the transporting comprises moving the cartridge and the magnetic field relative to each other.

32. The method of any one of claims 1-31, wherein the contacting comprises agitating a mixture comprising the sample and the first and second populations of magnetic particles.

33. The method of claim 32, wherein agitating comprises shaking the cartridge.

34. The method of any one of claims 13-33, wherein applying the magnetic force forms an aggregate of the first and second populations of magnetic particles which aggregate is spatially aligned with an entrance to the first channel.

35. The method of claim 34, wherein the entrance to the first channel comprises a tapered region that decreases in size from the first chamber to the first channel and facilitates transport of the aggregate from the first chamber to the second chamber via the first channel.

36. The method of claim 34 or 35, wherein the aggregate is spatially aligned with an entrance to the second channel.

37. The method of claim 36, wherein the entrance to the second channel comprises a tapered region that decreases in size from the second chamber to the second channel and facilitates transport of the aggregate from the second chamber to the third chamber via the second channel.

38. The method of any one of claims 13-37, wherein the transporting the first and second populations of magnetic particles from the first chamber to a second chamber of the cartridge by applying a magnetic force to the particles comprises placing a magnet adjacent the first chamber to cause formation of an aggregate comprising the magnetic particles, wherein the magnet is placed at a position such that the aggregate is spatially aligned with the entrance to the first and second channels.

39. The method of any one of claims 1-23, wherein the method is semi-automatic.

40. The method of any one of claims 1-23, wherein the step of contacting a sample with a first population of magnetic particles and a second population of magnetic particles in a first region of a sample processing cartridge comprises loading of the sample into the first chamber of the sample processing cartridge by a user or via a robot and wherein one or more of the remaining steps are carried out automatically by an instrument operably connected to the cartridge.

41. A sample processing cartridge comprising:
a first chamber, a second chamber, and a third chamber,
wherein the first chamber comprises a first population of magnetic particles and a second population of magnetic particles,
wherein the first chamber is fluidically connected to the second chamber via a first channel and the second chamber is fluidically connected to the third chamber via a second channel,
wherein the first population of magnetic particles is capable of associating with the target analyte, wherein optionally the target analyte comprises nucleic acids, and the magnetic particles in the second population are at least two-times larger in diameter than the first population of magnetic particles.

42. The sample processing cartridge according to claim 41, wherein the first population of magnetic particles and a second population of magnetic particles are present in a mixture.

43. The sample processing cartridge according to claim 41 or claim 42, wherein the first population of magnetic particles and a second population of magnetic particles are lyophilized.

44. The sample processing cartridge according to any one of claims 41-43, wherein the first population of magnetic particles and a second population of magnetic particles are located at an inlet to the first chamber, wherein flow of an aqueous solution through the inlet wets the magnetic particles and wherein subsequent agitation of the sample processing cartridge suspends the magnetic particles.

45. The sample processing cartridge according to claim 44, wherein the inlet to the first chamber comprises a compartment fluidically connected to the first chamber, wherein the compartment comprises the magnetic particles and wherein flow of an aqueous solution through compartment into the first chamber introduces the magnetic particles into the first chamber.

46. The sample processing cartridge according to any one of claims 41-45, wherein the magnetic particles in the first population have a diameter of 500 nm-10 um.

47. The sample processing cartridge according to any one of claims 41-46, wherein the magnetic particles in the second population have a size that is 2×-20× the size of the magnetic particles in the first population.

48. The sample processing cartridge according to any one of claims 41-47, wherein the second population of magnetic particles is incapable of associating with the target analyte, wherein optionally the target analyte comprises nucleic acids.

49. The sample processing cartridge according to any one of claims 41-48, wherein the sample processing cartridge is cylindrical and wherein the first, second, and third chambers are present on the outer wall of the cartridge.

50. A sample processing cartridge comprising:
a first chamber, an air-gap, a second chamber, an air chamber, and a third chamber,
wherein the air-gap is positioned between the first chamber and the second chamber and the air chamber is positioned between the second chamber and the third chamber, wherein the first chamber is fluidically connected to the second chamber via a first channel, wherein the air-gap extends through the first channel, the second chamber is fluidically connected to the air-chamber via a second channel, and the air chamber is fluidically connected to the third chamber via a third channel.

51. The sample processing cartridge according to claim 50, wherein the second chamber comprises two baffles placed at a position below the first and second channels, wherein the baffles are configured to decrease splashing of liquid present in the second chamber into the first and second channels.

52. The sample processing cartridge according to claim 50 or 51, wherein the third chamber comprises a shelf-baffle configured to allow magnetic beads to be transported, along a side wall, to a region below the shelf-baffle while decreasing splashing of liquid into the region below the shelf-baffle.

53. The sample processing cartridge according to any one of claims 50-52, wherein the first chamber comprises a first population of magnetic particles and a second population of magnetic particles, wherein the first population of magnetic particles is capable of associating with the target analyte, wherein optionally the target analyte comprises nucleic acids, and the magnetic particles in the second population are at least two-times larger in diameter than the first population of magnetic particles.

54. The sample processing cartridge according to any one of claims 50-53, wherein the first population of magnetic particles and a second population of magnetic particles are present in a mixture.

55. The sample processing cartridge according to any one of claims 50-54, wherein the first population of magnetic particles and a second population of magnetic particles are lyophilized.

56. The sample processing cartridge according to any one of claims 50-55, wherein the first population of magnetic particles and a second population of magnetic particles are located at an inlet to the first chamber, wherein flow of an aqueous solution through the inlet wets the magnetic particles and wherein subsequent agitation of the sample processing cartridge suspends the magnetic particles.

57. The sample processing cartridge according to any one of claims 50-56, wherein the inlet to the first chamber comprises a compartment fluidically connected to the first chamber, wherein the compartment comprises the magnetic particles and wherein flow of an aqueous solution through compartment into the first chamber introduces the magnetic particles into the first chamber.

58. The sample processing cartridge according to any one of claims 50-56, wherein the magnetic particles in the first population have a diameter of 500 nm-10 um.

59. The sample processing cartridge according to any one of claims 50-58, wherein the magnetic particles in the second population have a size that is 2×-20× the size of the magnetic particles in the first population.

60. The sample processing cartridge according to any one of claims 50-59, wherein the second population of magnetic particles is incapable of associating with the target analyte, wherein optionally the target analyte comprises nucleic acids.

61. The sample processing cartridge according to any one of claims 50-60, wherein the sample processing cartridge is cylindrical and wherein the first, second, and third chambers are present on the outer wall of the cartridge.

62. A system for processing a sample, the system comprising:
the sample processing cartridge according to any one of claims 41-61 and a magnet operably placed in association with the cartridge such that the magnet can exert a magnetic force on the magnetic particles.
